(19) United States
(12) Patent Application Publication
(10) Pub. No.: US 2026/0279867 A1
(43) Pub. Date: Sep. 17, 2026

(54) BIOLOGICAL BATTERY

(71) Applicant: Tahereh Safari, Qom (IR)

(72) Inventor: Tahereh Safari, Qom (IR)

(21) Appl. No.: 19/729,130

(22) Filed: May 4, 2026

Related U.S. Application Data
(63) Continuation-in-part of application No. PCT/IB2024/060852, filed on Nov. 3, 2024.

(30) Foreign Application Priority Data
Nov. 5, 2023 (IR) 14025014000300538
Nov. 3, 2024 (WO) PCT/IB2024/060852

(51) Int. Cl. H01M 8/16; A61N 1/378; H01M 4/38; H01M 4/62; H01M 6/04; H01M 6/38

(52) U.S. Cl. CPC H01M 8/16; H01M 4/38; H01M 4/628; H01M 6/045; H01M 6/385; A61N 1/3785

(57) ABSTRACT

In accordance with some embodiments herein, a battery is provided. The battery includes a plurality of electrodes, a set of electrical conductors configured to (i) connect one or more of the plurality of electrodes pairwise, and (ii) connect the plurality of electrodes to an electrical device, and a housing assembly configured to house an electrolyte and a set of electrodes. The electrolyte includes a bodily fluid and/or a natural material. The bodily fluid includes a blood, a cerebrospinal fluid, a urine, a body sweat, a peritoneal fluid, and/or an interstitial fluid. The natural material includes glucose, standard white vinegar, liquid copper ion, liquid iron ion, liquid zinc ion, fenugreek plant extract, melted white chocolate, liquid oil, flower petal water with bacteria, ice cream, heated and melted ice cream, dried soil, soil with water, cooked potato puree, lemon puree, sheep blood, peritoneal fluid, and/or cerebrospinal fluid.

### RELATED APPLICATION

This application claims priority to and is a continuation-in-part of International Application Number PCT/IB2024/060852, filed on Nov. 3, 2024, entitled “BIOLOGICAL BATTERY”, which claims priority to Iran Application Number 140250140003005389, filed on Nov. 5, 2023. International Application Number PCT/IB2024/060852 and Iran Application Number 140250140003005389 are incorporated herein by reference in their entirety.

### BACKGROUND

A battery includes one or more electrochemical cells that convert stored chemical energy into electrical energy, and may have a positive terminal and a negative terminal. Electrolytes in an electrochemical cell may allow movement of ions between electrodes to facilitate generation of current supplied by the battery to perform work.

### DESCRIPTION OF THE DRAWINGS

While the techniques presented herein may be embodied in alternative forms, the particular embodiments illustrated in the drawings are only a few examples that are supplemental of the description provided herein. These embodiments are not to be interpreted in a limiting manner, such as limiting the claims appended hereto.

FIG. 1A illustrates a side view of a battery, highlighting its various cells, according to some embodiments.

FIG. 1B illustrates a view of different components of a battery, according to some embodiments.

FIG. 1C illustrates movable sections of a battery, according to some embodiments.

FIG. 1D illustrates a 2-dimensional view of a battery comprising a plurality of buttons associated with a plurality of cells related to the battery, according to some embodiments.

FIG. 1E illustrates a 2-dimensional view of a battery comprising a plurality of buttons associated with a plurality of cells related to the battery, wherein the plurality of buttons are pressed down and immersed in a plurality of electrolytes inside a housing assembly, according to some embodiments.

FIG. 1F presents a table of voltage, amperage, and power values generated by a 4-cell battery with different electrodes immersed in a lemon puree electrolyte with the same wetted surfaces of the electrodes, according to some embodiments.

FIG. 1G presents a table of voltage, amperage, and power values generated by a 4-cell battery with different electrodes immersed in a standard white vinegar electrolyte with the same wetted surfaces of the electrodes, according to some embodiments.

FIG. 1H presents a table of voltage, amperage, and power values generated by a 4-cell battery and an 8-cell battery with copper anodes and zinc cathodes immersed in a standard white vinegar electrolyte with the same wetted surfaces of the copper anodes and the zinc cathodes, according to some embodiments.

FIG. 1I presents a table of voltage, amperage, and power values generated by a 4-cell battery with copper anodes and zinc cathodes immersed in a standard white vinegar electrolyte with different wetted surfaces of the copper anodes and the zinc cathodes, according to some embodiments.

FIG. 1J presents a table of voltage, amperage, and power values generated by a 1-cell battery, a 3-cell battery, a 5-cell battery, a 7-cell battery, a 10-cell battery, a 13-cell battery and a 19-cell battery with copper anodes and zinc cathodes immersed in a sheep blood electrolyte with the same wetted surfaces of the copper anodes and the zinc cathodes, according to some embodiments.

FIG. 2 illustrates a needle patch battery, according to some embodiments.

FIG. 3A illustrates a needle patch battery attached to a person's neck, and a wire connects it to a hearing aid, according to some embodiments.

FIG. 3B illustrates a plurality of needle patch batteries attached to various areas of a body of a person (e.g., a patient), according to some embodiments.

FIG. 4 illustrates a plurality of medical applications of a battery (e.g., a needle patch battery) within a plurality of implantable medical devices, according to some embodiments.

### DETAILED DESCRIPTION

The following subject matter may be embodied in a variety of different forms, such as methods, compositions, materials, and/or systems.

Accordingly, this subject matter is not intended to be construed as limited to any example embodiments set forth herein. Rather, example embodiments are provided merely to be illustrative.

Where a range of values is provided, it is understood that each intervening value, to the tenth of the unit of the lower limit unless the context clearly dictates otherwise, between the upper and lower limit of that range and any other stated or intervening value in that stated range is encompassed within the disclosure. Ranges from any lower limit to any upper limit are contemplated. The upper and lower limits of these smaller ranges which may independently be included in the smaller ranges is also encompassed within the disclosure, subject to any specifically excluded limit in the stated range. Where the stated range includes one or both of the limits, ranges excluding either both of those included limits are also included in the disclosure.

Although any methods and/or materials similar or equivalent to those described herein can also be used in the practice or testing of the present disclosure, the preferred methods and materials are now described. All publications mentioned herein are incorporated herein by reference to disclose and described the methods and/or materials in connection with which the publications are cited.

It must be noted that as used herein and in the appended claims, the singular forms “a”, “and”, and “the” include plural references unless the context clearly dictates otherwise.

Unless otherwise defined, all technical and scientific terms used herein have the same meaning as commonly understood by one of ordinary skill in the art to which this disclosure belongs. The terminology used in the description of the disclosure herein is for describing particular embodiments only and is not intended to be limiting of the disclosure. All publications, patent applications, patents, figures and other references mentioned herein are expressly incorporated by reference in their entirety.

Conventional batteries and/or bio-fuel cells designed for biological or medical applications face significant limitations that hinder their widespread practical use. Existing technologies frequently rely on expensive noble metals, carbon nanotubes, specialized enzymes, or complex nanostructures, which substantially increase manufacturing costs and complicate large-scale production. Moreover, many such systems suffer from poor long-term stability, enzyme degradation, catalyst poisoning, limited biocompatibility, and insufficient electrical conductivity, resulting in low power output and reduced operational lifetime. Previously disclosed bio-fuel cells and biological batteries, including those powered by glucose or blood, typically generate only minimal voltage and current densities, often in the nanowatt to milliwatt range. As a result, they are inadequate for reliably powering electronic or medical devices. In addition, many prior solutions require invasive surgical implantation, complex electrode architectures, or the use of membranes and coatings that further increase system complexity and risk, rendering them impractical for safe, cost-effective, and non-invasive applications. The present disclosure overcomes these limitations by providing a novel biological and environmentally friendly battery system that utilizes biological materials as functional electrochemical components. Unlike prior art systems, the disclosed battery does not depend on costly noble metals, unstable enzymes, or chemically fragile nanostructures. Instead, it employs a plurality of electrodes arranged in an efficient configuration, electrically connected through first and second sets of conductors, and housed within a structure designed to contain a suitable electrolyte. Advantageously, the disclosed battery enables locally distributed power generation, improved electrical performance, enhanced chemical stability, and reduced production cost, while maintaining high biocompatibility. The system is capable of producing useful electrical energy as well as beneficial chemical products such as anions, cations, and organic acids under controlled electrochemical conditions.

Furthermore, the battery can be deployed externally, for example as a skin-mounted device, thereby eliminating the need for invasive implantation procedures. Accordingly, the present disclosure provides a practical, scalable, and cost-effective alternative to existing biological batteries and bio-fuel cells, addressing the longstanding challenges of low power output, high cost, limited stability, and poor biocompatibility encountered in the prior art.

The present disclosure provides a battery (e.g., a biological battery). The battery includes a plurality of electrodes, a first set of electrical conductors, a second set of electrical conductors, and/or a housing assembly. The first set of electrical conductors are configured to connect one or more of the plurality of electrodes pairwise. The second set of electrical conductors are configured to connect the plurality of electrodes to an electrical device. The housing assembly is configured to house an electrolyte and/or a set of electrodes of the plurality of electrodes.

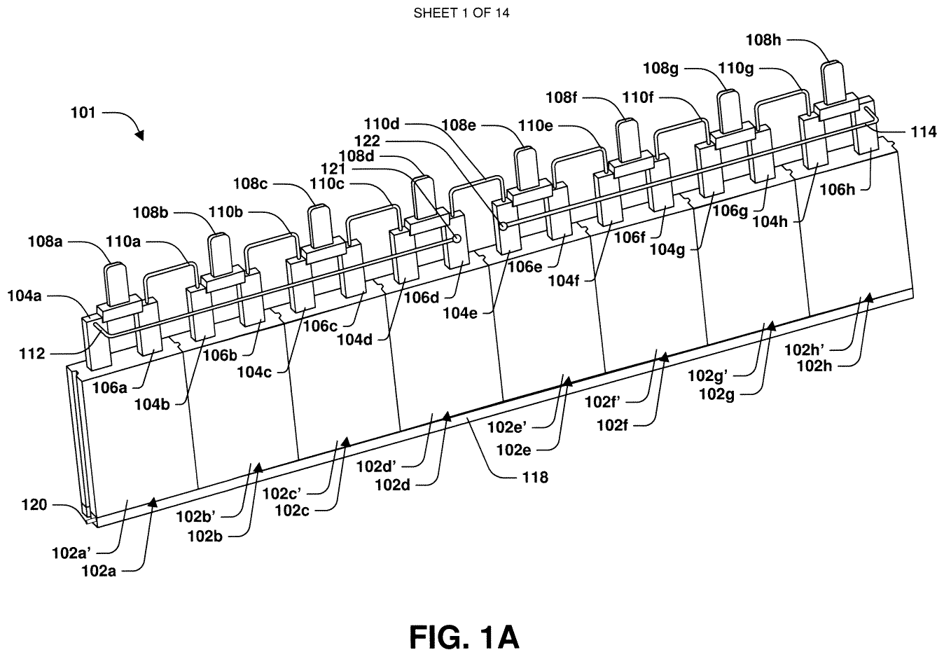

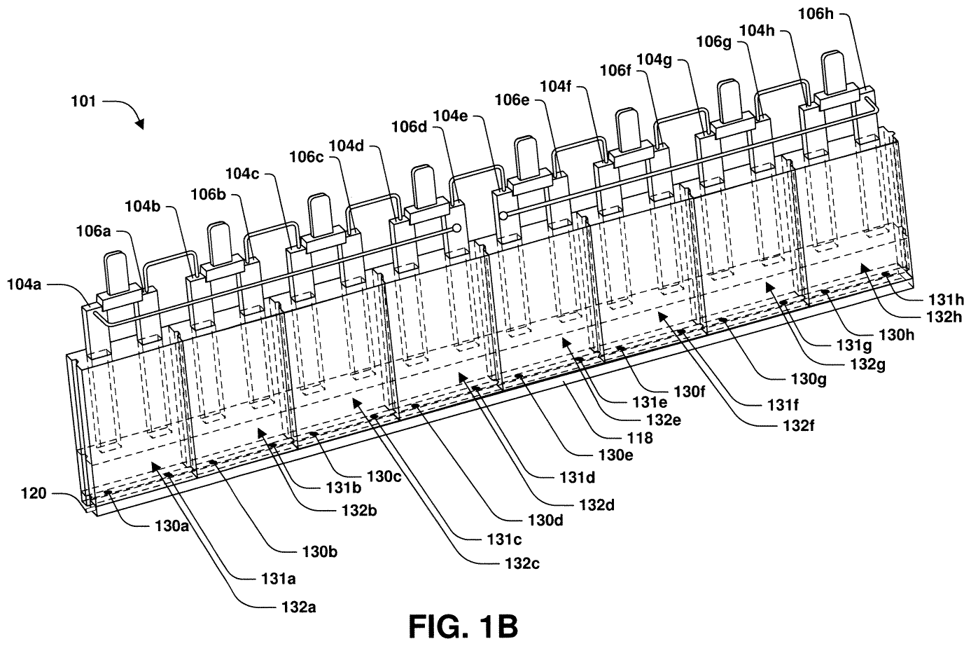

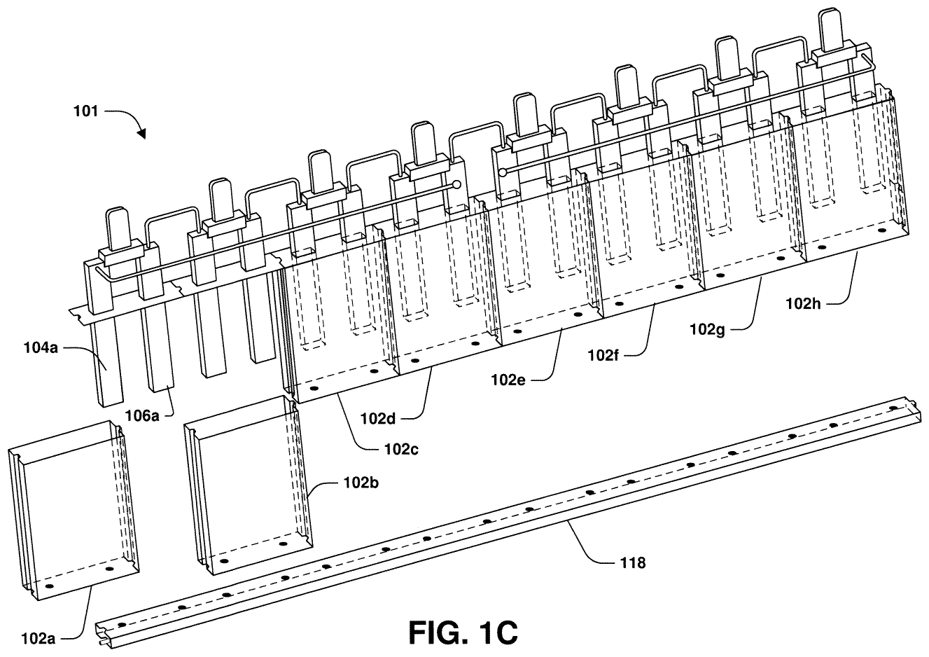

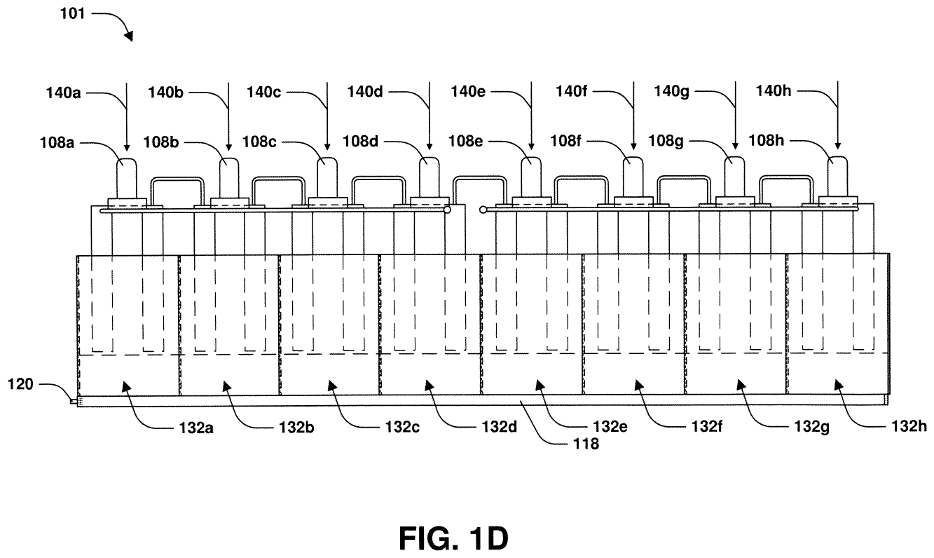

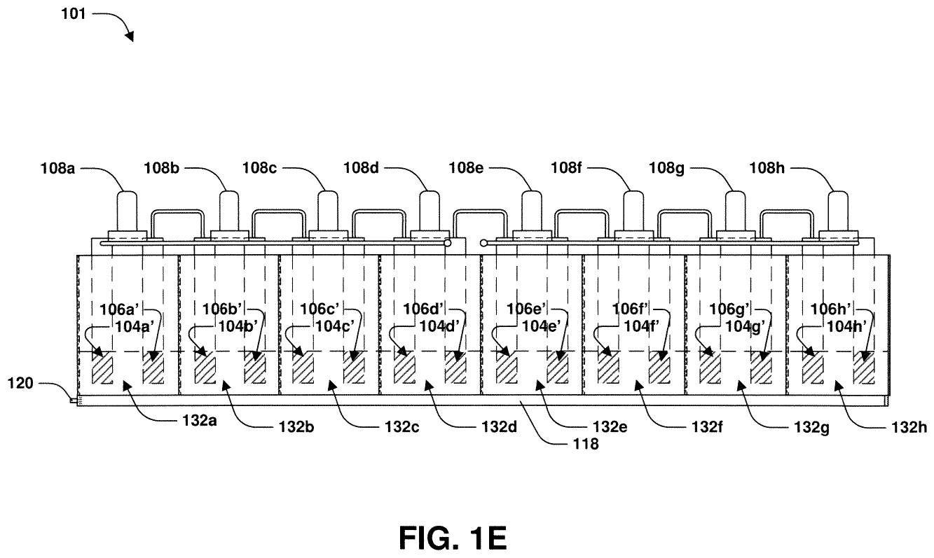

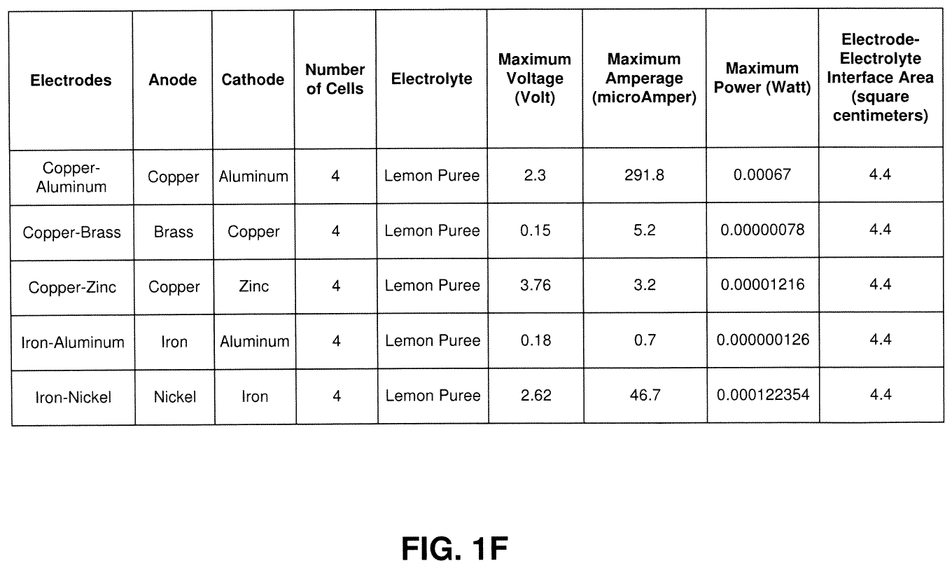

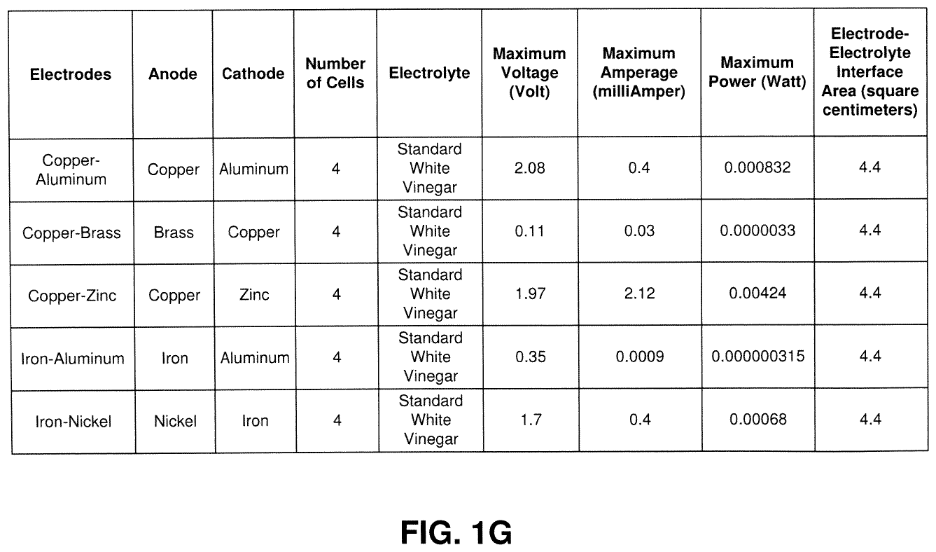

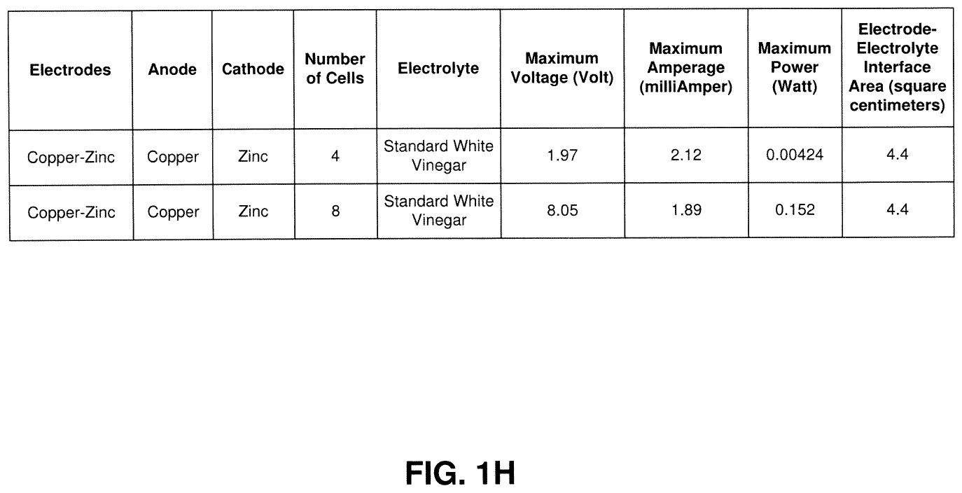

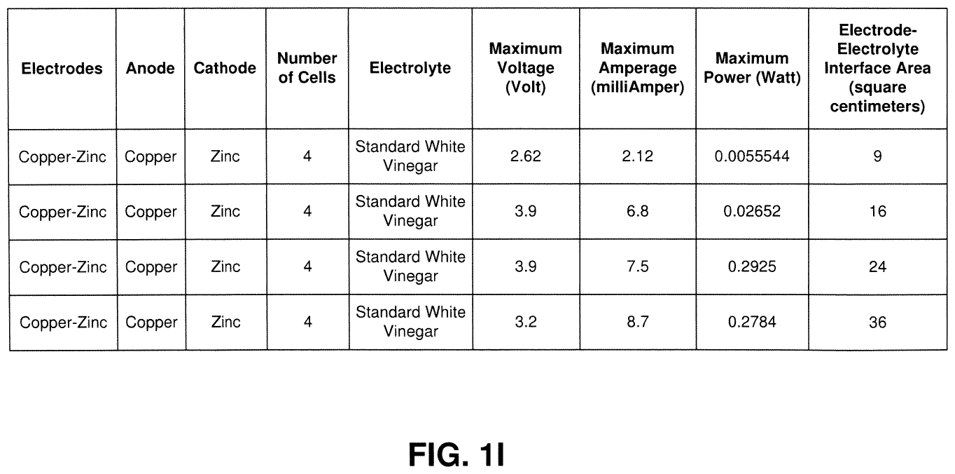

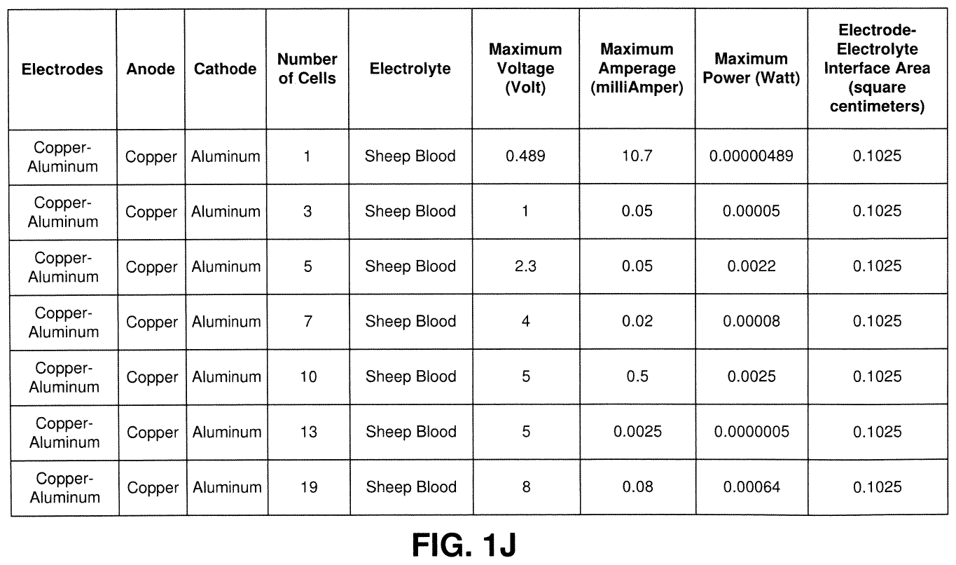

FIGS. 1A-1E illustrate different views of a battery 101, according to some embodiments. FIG. 1A illustrates a side view of the battery 101, highlighting its various cells, according to some embodiments. In some examples, the battery 101 may be a biological battery and may comprise one or more cells (e.g., a first cell 102a, a second cell 102b, a third cell 102c, a fourth cell 102d, a fifth cell 102e, a sixth cell 102f, a seventh cell 102g, and/or an eighth cell 102h).

In some examples, the battery 101 may comprise a plurality of electrodes, wherein the plurality of electrodes may comprise a plurality of anodes (e.g., a first anode 104a, a second anode 104b, a third anode 104c, a fourth anode 104d, a fifth anode 104e, a sixth anode 104f, a seventh anode 104g, and/or an eighth anode 104h) and/or a plurality of cathodes (e.g., a first cathode 106a, a second cathode 106b, a third cathode 106c, a fourth cathode 106d, a fifth cathode 106e, a sixth cathode 106f, a seventh cathode 106g, and/or an eighth cathode 106h).

In some examples, the battery 101 may comprise a set of electrical conductors, wherein the set of electrical conductors may comprise a first set of electrical conductors (a second electrical conductor 110a, a third electrical conductor 110b, a fourth electrical conductor 110c, a fifth electrical conductor 110d, a sixth electrical conductor 110e, a seventh electrical conductor 110f, and/or an eighth electrical conductor 110g) and/or a second set of electrical conductors (e.g., a first electrical conductor 112, and/or a ninth electrical conductor 114). In some examples, the first electrical conductor 112 may comprise a head 121 and/or the ninth electrical conductor 114 may comprise a head 122. In some examples, the head 121 may be a positive terminal of the battery 101 and the head 122 may be a negative terminal of the battery 101. In some examples, the head 121 may be a negative terminal of the battery 101 and the head 122 may be a positive terminal of the battery 101. In some examples, the first set of electrical conductors may be configured to connect one or more of the plurality of electrodes pairwise. In some examples, the second set of electrical conductors may be configured to connect the plurality of electrodes to one or more electrical devices. In some examples, the second electrical conductor 110a may connect the first cathode 106a to the second anode 104b. In some examples, the third electrical conductor 110b may connect the second cathode 106b to the third anode 104c. In some examples, the fourth electrical conductor 110c may connect the third cathode 106c to the fourth anode 104d. In some examples, the fifth electrical conductor 110d may connect the fourth cathode 106d to the fifth anode 104e. In some examples, the sixth electrical conductor 110e may connect the fifth cathode 106e to the sixth anode 104f. In some examples, the seventh electrical conductor 110f may connect the sixth cathode 106f to the seventh anode 104g. In some examples, the eighth electrical conductor 110g may connect the seventh cathode 106g to the eighth anode 104h. In some examples, the first electrical conductor 112 may be connected to the first anode 104a on one side and the other side of the first electrical conductor 112 may be a positive terminal of the battery 101.

In some examples, the first electrical conductor 112 may be connected to the first anode 104a on one side and the other side of the first electrical conductor 112 may be a negative terminal of the battery 101. The ninth electrical conductor 114 may be connected to the eighth cathode 106h on one side and the other side of the ninth electrical conductor 114 may be a negative terminal of the battery 101. In some examples, the ninth electrical conductor 114 may be connected to the eighth cathode 106h on one side and the other side of the ninth electrical conductor 114 may be a positive terminal of the battery 101.

In some examples, the set of electrical conductors may be a metal wire (e.g., a copper wire). In some examples, the battery 101 may comprise a housing assembly (e.g., a plurality of housings, a first housing 102a′, a second housing 102b′, a third housing 102c′, a fourth housing 102d′, a fifth housing 102e′, a sixth housing 102f′, a seventh housing 102g′, and/or an eighth housing 102h′) to house one or more electrolytes and/or a set of electrodes of the plurality of electrodes.

In some examples, the housing assembly may comprise an enclosure 118 (e.g., a reservoir) connected to the plurality of housings, wherein the enclosure 118 may be configured to allow the one or more electrolytes to enter the plurality of housings. The enclosure 118 may comprise an inlet port 120 configured to receive the one or more electrolytes and conduct the one or more electrolytes to the enclosure 118.

As shown in FIG. 1, the battery 101 is an 8-cell battery, wherein each cell of the 8-cell battery comprises at least one anode and one cathode. The first cell 102a may comprise the first housing 102a′, the first anode 104a, the first cathode 106a and/or a first button 108a. The second cell 102b may comprise the second housing 102b′, the second anode 104b, the second cathode 106b and/or a second button 108b. The third cell 102c may comprise the third housing 102c′, the third anode 104c, the third cathode 106c and/or a third button 108c. The fourth cell 102d may comprise the fourth housing 102d′, the fourth anode 104d, the fourth cathode 106d and/or a fourth button 108d. The fifth cell 102e may comprise the fifth housing 102e′, the fifth anode 104e, the fifth cathode 106e and/or a fifth button 108e. The sixth cell 102f may comprise the sixth housing 102f′, the sixth anode 104f, the sixth cathode 106f and/or a sixth button 108f. The seventh cell 102g may comprise the seventh housing 102g′, the seventh anode 104g, the seventh cathode 106g and/or a seventh button 108g. The eighth cell 102h may comprise the eighth housing 102h′, the eighth anode 104h, the eighth cathode 106h and/or an eighth button 108h. Although FIG. 1A shows eight cells for the battery 101, any number of cells are contemplated in the present disclosure.

In some examples, the plurality of housings may be made of one or more materials that are electrically insulating. In some examples, the first anode 104a may be connected to the first cathode 106a via the first button 108a, the second anode 104b may be connected to the second cathode 106b via the second button 108b, the third anode 104c may be connected to the third cathode 106c via the third button 108c, the fourth anode 104d may be connected to the fourth cathode 106d via the fourth button 108d, the fifth anode 104e may be connected to the fifth cathode 106e via the fifth button 108e, the sixth anode 104f may be connected to the sixth cathode 106f via the sixth button 108f, the seventh anode 104g may be connected to the seventh cathode 106g via the seventh button 108g and/or the eighth anode 104h may be connected to the eighth cathode 106h via the eighth button 108h. In some examples, the first button 108a, the second button 108b, the third button 108c, the fourth button 108d, the fifth button 108e, the sixth button 108f, the seventh button 108g and/or the eighth button 108h may be made of one or more materials that are electrically insulating. In some examples, the first button 108a, the second button 108b, the third button 108c, the fourth button 108d, the fifth button 108e, the sixth button 108f, the seventh button 108g and/or the eighth button 108h may be made of one or more materials that are electrically conductive.

In some examples, the first housing 102a′ may be configured to house a first electrolyte, the first anode 104a and/or the first cathode 106a. In some examples, the second housing 102b′ may be configured to house a second electrolyte (e.g., the first electrolyte), the second anode 104b and/or the second cathode 106b. In some examples, the third housing 102c′ may be configured to house a third electrolyte (e.g., the first electrolyte), the third anode 104c and/or the third cathode 106c. In some examples, the fourth housing 102d′ may be configured to house a fourth electrolyte (e.g., the first electrolyte), the fourth anode 104d and/or the fourth cathode 106d. In some examples, the fifth housing 102e′ may be configured to house a fifth electrolyte (e.g., the first electrolyte), the fifth anode 104e and/or the fifth cathode 106e. In some examples, the sixth housing 102f′ may be configured to house a sixth electrolyte (e.g., the first electrolyte), the sixth anode 104f and/or the sixth cathode 106f. In some examples, the seventh housing 102g′ may be configured to house a seventh electrolyte (e.g., the first electrolyte), the seventh anode 104g and/or the seventh cathode 106g. In some examples, the eighth housing 102h′ may be configured to house an eighth electrolyte (e.g., the first electrolyte), the eighth anode 104h and/or the eighth cathode 106h.

In some examples, an anode (e.g., the first anode 104a) of the plurality of electrodes and/or a cathode (e.g., the eighth cathode 106h) of the plurality of electrodes may undergo electrochemical reactions with the one or more electrolytes in the housing assembly to generate electrical energy for the one or more electrical devices.

In some examples, the one or more electrical devices may comprise a mobile charger, a mobile phone, a tablet, a laptop and/or a medical device. In some examples, the medical device may comprise a hearing aid, a cochlear implant, a pacemaker, an artificial heart, an insulin pump, a drug pump, a retinal implant and/or a nerve stimulator.

In some examples, the plurality of anodes may be made of Copper, Brass, Platinum, Gold, Silver, Palladium, Lithium, Graphite, Zinc, Aluminum, Iron and/or Nickel.

In some examples, the plurality of cathodes may be made of Copper, Brass, Platinum, Gold, Silver, Palladium, Lithium, Graphite, Zinc, Aluminum, Iron and/or Nickel.

In some examples, at least a portion of the plurality of electrodes in the battery 101 may be covered with a magnesium material to prevent corrosion of the plurality of electrodes and/or to maintain conductivity and electron transfer of the plurality of electrodes.

In some examples, the one or more electrolytes may comprise one or more bodily fluids. The one or more bodily fluids may comprise a blood (e.g., a human blood, an animal blood, etc.), a cerebrospinal fluid (e.g., a human cerebrospinal fluid, an animal cerebrospinal fluid, etc.), a urine (e.g., a human urine, an animal urine, etc.), a body sweat (e.g., a human body sweat, an animal body sweat, etc.), a peritoneal fluid (e.g., a human peritoneal fluid, an animal peritoneal fluid, etc.) and/or an interstitial fluid (e.g., a human interstitial fluid, an animal interstitial fluid, etc.).

In some examples, the one or more electrolytes may comprise one or more natural materials. The one or more natural materials may comprise glucose, standard white vinegar, liquid copper ion, liquid iron ion, liquid zinc ion, fenugreek plant extract, melted white chocolate, liquid oil, flower petal water with bacteria, ice cream, heated and melted ice cream, dried soil, soil with water (hard clay), soil with water (very thin clay), cooked potato puree, lemon puree, sheep blood, peritoneal fluid and/or cerebrospinal fluid. In some examples, the one or more natural materials may comprise one or more natural enzymes.

In some examples, the battery 101 may be an enzymatic fuel cell. In some examples, the battery 101 may be an enzymatic bio-fuel cell.

FIG. 1B illustrates a view of different components of the battery 101, according to some embodiments. As shown in FIG. 1B, the enclosure 118 may allow (i) the first electrolyte (shown with reference number 132a) to enter the first housing 102a′ via a hole 130a and/or a hole 131a, (ii) the second electrolyte (shown with reference number 132b) to enter the second housing 102b′ via a hole 130b and/or a hole 131b, (iii) the third electrolyte (shown with reference number 132c) to enter the third housing 102c′ via a hole 130c and/or a hole 131c, (iv) the fourth electrolyte (shown with reference number 132d) to enter the fourth housing 102d′ via a hole 130d and/or a hole 131d, (v) the fifth electrolyte (shown with reference number 132e) to enter the fifth housing 102e′ via a hole 130e and/or a hole 131e, (vi) the sixth electrolyte (shown with reference number 132f) to enter the sixth housing 102f′ via a hole 130f and/or a hole 131f, (vii) the seventh electrolyte (shown with reference number 132g) to enter the seventh housing 102g′ via a hole 130g and/or a hole 131g, and (viii) the eighth electrolyte (shown with reference number 132h) to enter the eighth housing 102h′ via a hole 130h and/or a hole 131h.

Although FIG. 1B shows two holes for each cell of the battery 101, any number of holes are contemplated in the present disclosure.

FIG. 1C illustrates movable sections of the battery 101, according to some embodiments. As shown in FIG. 1C, each cell is attached to its adjacent cell through one or more rails, forming a continuous structure. Each housing may be separated from the battery 101 and reattached to its adjacent housing. In some examples, the enclosure 118 may be separated from the plurality of housings and reattached to the plurality of housings. In some examples, all parts of the battery 101 may easily be fixed or replaced by new parts. Although FIG. 1C shows sixteen holes for the enclosure 118, any number of holes are contemplated in the present disclosure.

FIG. 1D illustrates a 2-dimensional view of the battery 101 comprising a plurality of buttons (e.g., the first button 108a, the second button 108b, the third button 108c, the fourth button 108d, the fifth button 108e, the sixth button 108f, the seventh button 108g, the eighth button 108h and/or a plurality of switches) associated with a plurality of cells (e.g., the first cell 102a, the second cell 102b, the third cell 102c, the fourth cell 102d, the fifth cell 102e, the sixth cell 102f, the seventh cell 102g and/or the eighth cell 102h) related to the battery 101, according to some embodiments. As shown in FIG. 1D, the plurality of electrodes are not in contact with the one or more electrolytes inside the housing assembly. In some examples, pressing each button may cause the battery 101 to generate a different output voltage, a different output amperage and/or a different output power. For example, when one button is pressed, a first output voltage, a first output amperage and/or a first output power may be generated by the battery 101 and when all buttons are pressed, a second output voltage, a second output amperage and/or a second output power may be generated by the battery 101.

FIG. 1E illustrates a 2-dimensional view of the battery 101 comprising the plurality of buttons associated with the plurality of cells related to the battery 101, wherein the plurality of buttons are pressed down and the plurality of electrodes are immersed in a plurality of electrolytes inside the housing assembly, according to some embodiments. In some examples, the first button 108a may be pressed in a direction 140a, the second button 108b may be pressed in a direction 140b, the third button 108c may be pressed in a direction 140c, the fourth button 108d may be pressed in a direction 140d, the fifth button 108e may be pressed in a direction 140e, the sixth button 108f may be pressed in a direction 140f, the seventh button 108g may be pressed in a direction 140g, the eighth button 108h may be pressed in a direction 140h.

As shown in FIG. 1E, the plurality of electrodes are in contact with the one or more electrolytes inside the housing assembly. After immersing the plurality of electrodes inside the one or more electrolytes, at least a portion of each electrode of the plurality of electrodes may sink into the one or more electrolytes and may get wet. In some examples, the battery 101 may be in a first mode or in a second mode. For example, when the plurality of electrodes are not in contact with the one or more electrolytes, the battery 101 may be in the first mode which is an Off-mode (e.g., de-active mode) and when the plurality of electrodes are in contact with the one or more electrolytes, the battery 101 may be in the second mode which is an On-mode (e.g., active mode). In some examples, the first anode 104a may comprise a wetted surface 104a′, the second anode 104b may comprise a wetted surface 104b′, the third anode 104c may comprise a wetted surface 104c′, the fourth anode 104d may comprise a wetted surface 104d′, the fifth anode 104e may comprise a wetted surface 104e′, the sixth anode 104f may comprise a wetted surface 104f′, the seventh anode 104g may comprise a wetted surface 104g′ and/or the eighth anode 104h may comprise a wetted surface 104h′. In some examples, the first cathode 106a may comprise a wetted surface 106a′, the second cathode 106b may comprise a wetted surface 106b′, the third cathode 106c may comprise a wetted surface 106c′, the fourth cathode 106d may comprise a wetted surface 106d′, the fifth cathode 106e may comprise a wetted surface 106e′, the sixth cathode 106f may comprise a wetted surface 106f′, the seventh cathode 106g may comprise a wetted surface 106g′ and/or the eighth cathode 106h may comprise a wetted surface 106h′.

In some examples, a user may select a desired voltage and/or a desired amperage (e.g., a desired current) based upon a power requirement associated with a device. In some examples, a voltage and/or an amperage associated with the device may be completely customizable via selecting number of the plurality of buttons. As shown in FIG. 1D, no button is activated, and no electrode is immersed in an electrolyte. By activating the plurality of buttons, one or more levers may move down, immersing at least two connected electrodes in an electrolyte inside a housing, thus activating the battery 101 with a corresponding number of cells and generating an electric current. In some examples, each button of the plurality of buttons may show a corresponding voltage value. For example, upon pushing the first button 108a, the battery 101 produces a first voltage (e.g., 1 volt). For example, upon pushing the first button 108a and the second button 108b, the battery 101 produces a second voltage (e.g., 2 volts). For example, upon pushing the first button 108a, the second button 108b and the third button 108c, the battery 101 produces a third voltage (e.g., 3 volts). In some examples, the battery 101 may generate a voltage range between about 1 volt to about 25 volts. In some examples, the battery 101 may generate a voltage range between about 0.001 volts to about 220 volts or more. In some examples, the battery 101 may utilize a voltage booster module to increase a quantity of voltage level.

FIG. 1F presents a table of voltage, amperage, and power values generated by a 4-cell battery with different electrodes immersed in a lemon puree electrolyte with the same wetted surfaces (e.g., electrode-electrolyte interface area) of the electrodes, according to some embodiments. For example, a 4-cell battery with a Copper anode for each cell, an Aluminum cathode for each cell and a lemon puree electrolyte for each cell, which has electrodes each with a wetted surface of 4.4 square centimeters, may generate 2.3 volts, 291.8 microampere and 0.00067 watt.

For example, a 4-cell battery with a Brass anode for each cell, a Copper cathode for each cell and a lemon puree electrolyte for each cell, which has electrodes each with a wetted surface of 4.4 square centimeters, may generate 0.15 volts, 5.2 microampere and 0.00000078 watt.

For example, a 4-cell battery with a Copper anode for each cell, a Zinc cathode for each cell and a lemon puree electrolyte for each cell, which has electrodes each with a wetted surface of 4.4 square centimeters, may generate 3.76 volts, 3.2 microampere and 0.00001216 watt.

For example, a 4-cell battery with an Iron anode for each cell, an Aluminum cathode for each cell and a lemon puree electrolyte for each cell, which has electrodes each with a wetted surface of 4.4 square centimeters, may generate 0.18 volts, 0.7 microampere and 0.000000126 watt.

For example, a 4-cell battery with a Nickel anode for each cell, an Iron cathode for each cell and a lemon puree electrolyte for each cell, which has electrodes each with a wetted surface of 4.4 square centimeters, may generate 2.62 volts, 46.7 microampere and 0.000122354 watt. Choosing type of anode, cathode and/or electrolyte may affect current generation (e.g., current value, voltage value, amperage value, power value).

FIG. 1G presents a table of voltage, amperage, and power values generated by a 4-cell battery with different electrodes immersed in a standard white vinegar electrolyte with the same wetted surfaces (e.g., electrode-electrolyte interface area) of the electrodes, according to some embodiments.

For example, a 4-cell battery with a Copper anode for each cell, an Aluminum cathode for each cell and a standard white vinegar electrolyte for each cell, which has electrodes each with a wetted surface of 4.4 square centimeters, may generate a maximum voltage of 2.08 volts, a maximum amperage of 0.4 milliampere and a maximum power of 0.000832 watt.

For example, a 4-cell battery with a Brass anode for each cell, a Copper cathode for each cell and a standard white vinegar electrolyte for each cell, which has electrodes each with a wetted surface of 4.4 square centimeters, may generate a maximum voltage of 0.11 volts, a maximum amperage of 0.03 milliampere and a maximum power of 0.0000033 watt.

For example, a 4-cell battery with a Copper anode for each cell, a Zinc cathode for each cell and a standard white vinegar electrolyte for each cell, which has electrodes each with a wetted surface of 4.4 square centimeters, may generate a maximum voltage of 1.97 volts, a maximum amperage of 2.12 milliampere and a maximum power 0.00424 watt.

For example, a 4-cell battery with an Iron anode for each cell, an Aluminum cathode for each cell and a standard white vinegar electrolyte for each cell, which has electrodes each with a wetted surface of 4.4 square centimeters, may generate a maximum voltage of 0.35 volts, a maximum amperage 0.0009 of milliampere and a maximum power of 0.000000315 watt.

For example, a 4-cell battery with a Nickel anode for each cell, an Iron cathode for each cell and a standard white vinegar electrolyte for each cell, which has electrodes each with a wetted surface of 4.4 square centimeters, may generate a maximum voltage of 1.7 volts, a maximum amperage of 0.4 milliampere and a maximum power of 0.00068 watt. Choosing type of anode, cathode, electrolyte and/or type of electrolyte may affect current generation (e.g., current value, voltage value, amperage value, power value).

FIG. 1H presents a table of voltage, amperage, and power values generated by a 4-cell battery and an 8-cell battery with copper anodes and zinc cathodes immersed in a standard white vinegar electrolyte with the same wetted surfaces of the copper anodes and the zinc cathodes, according to some embodiments. For example, the 4-cell battery with a Copper anode for each cell, a Zinc cathode for each cell and a standard white vinegar electrolyte for each cell, which has electrodes each with a wetted surface of 4.4 square centimeters, may generate a maximum voltage of 1.97 volts, a maximum amperage of 2.12 milliampere and a maximum power of 0.00424 watt.

For example, the 8-cell battery with a Copper anode for each cell, a Zinc cathode for each cell and a standard white vinegar electrolyte for each cell, which has electrodes each with a wetted surface of 4.4 square centimeters, may generate a maximum voltage of 8.05 volts, a maximum amperage of 1.89 milliampere and a maximum power of 0.152 watt. Choosing type of anode, cathode, electrolyte and/or number of cells may affect current generation (e.g., current value, voltage value, amperage value, power value).

FIG. 1I presents a table of voltage, amperage, and power values generated by a 4-cell battery with copper anodes and zinc cathodes immersed in a standard white vinegar electrolyte with different wetted surfaces of the copper anodes and the zinc cathodes, according to some embodiments. For example, a 4-cell battery with a Copper anode for each cell, a Zinc cathode for each cell and a standard white vinegar electrolyte for each cell, which has electrodes each with a wetted surface of 9 square centimeters, may generate a maximum voltage of 2.62 volts, a maximum amperage of 2.12 milliampere and a maximum power of 0.0055544 watt.

For example, a 4-cell battery with a Copper anode for each cell, a Zinc cathode for each cell and a standard white vinegar electrolyte for each cell, which has electrodes each with a wetted surface of 16 square centimeters, may generate a maximum voltage of 3.9 volts, a maximum amperage of 6.8 milliampere and a maximum power of 0.02652 watt.

For example, a 4-cell battery with a Copper anode for each cell, a Zinc cathode for each cell and a standard white vinegar electrolyte for each cell, which has electrodes each with a wetted surface of 24 square centimeters, may generate a maximum voltage of 3.9 volts, a maximum amperage of 7.5 milliampere and a maximum power of 0.2925 watt.

For example, a 4-cell battery with a Copper anode for each cell, a Zinc cathode for each cell and a standard white vinegar electrolyte for each cell, which has electrodes each with a wetted surface of 36 square centimeters, may generate a maximum voltage of 3.2 volt, a maximum amperage of 8.7 milliampere and a maximum power of 0.2784 watt. Choosing type of anode, cathode, wetted surface of each anode, wetted surface of each cathode, type of electrolyte and/or number of cells may affect current generation (e.g., current value, voltage value, amperage value, power value).

FIG. 1J presents a table of voltage, amperage, and power values generated by a 1-cell battery, a 3-cell battery, a 5-cell battery, a 7-cell battery, a 10-cell battery, a 13-cell battery and a 19-cell battery with copper anodes and Aluminum cathodes immersed in a sheep blood electrolyte with the same wetted surfaces of the copper anodes and the Aluminum cathodes, according to some embodiments.

For example, a 1-cell battery with a Copper anode for each cell, an Aluminum cathode for each cell and a sheep blood electrolyte for each cell, which has electrodes each with a wetted surface of 0.1025 square centimeters, may generate a maximum voltage of 0.489 volts, a maximum amperage of 10.7 milliampere and a maximum power of 0.00000489 watt.

For example, a 3-cell battery with a Copper anode for each cell, an Aluminum cathode for each cell and a sheep blood electrolyte for each cell, which has electrodes each with a wetted surface of 0.1025 square centimeters, may generate a maximum voltage of 1 volt, a maximum amperage of 0.05 milliampere and a maximum power of 0.00005 watt.

For example, a 5-cell battery with a Copper anode for each cell, an Aluminum cathode for each cell and a sheep blood electrolyte for each cell, which has electrodes each with a wetted surface of 0.1025 square centimeters, may generate a maximum voltage of 2.3 volts, a maximum amperage of 0.05 milliampere and a maximum power of 0.0022 watt.

For example, a 7-cell battery with a Copper anode for each cell, an Aluminum cathode for each cell and a sheep blood electrolyte for each cell, which has electrodes each with a wetted surface of 0.1025 square centimeters, may generate a maximum voltage of 4 volts, a maximum amperage of 0.02 milliampere and a maximum power of 0.00008 watt.

For example, a 10-cell battery with a Copper anode for each cell, an Aluminum cathode for each cell and a sheep blood electrolyte for each cell, which has electrodes each with a wetted surface of 0.1025 square centimeters, may generate a maximum voltage of 5 volts, a maximum amperage of 0.5 milliampere and a maximum power of 0.0025 watt.

For example, a 13-cell battery with a Copper anode for each cell, an Aluminum cathode for each cell and a sheep blood electrolyte for each cell, which has electrodes each with a wetted surface of 0.1025 square centimeters, may generate a maximum voltage of 5 volts, a maximum amperage of 0.0025 milliampere and a maximum power of 0.0000005 watt.

For example, a 19-cell battery with a Copper anode for each cell, an Aluminum cathode for each cell and a sheep blood electrolyte for each cell, which has electrodes each with a wetted surface of 0.1025 square centimeters, may generate a maximum voltage of 8 volts, a maximum amperage of 0.08 milliampere and a maximum power of 0.00064 watt. Choosing type of anode, cathode, electrolyte and/or number of cells may affect current generation (e.g., current value, voltage value, amperage value, power value).

In some examples, the battery 101 may generate an ascending power in a first period of time and a constant power in a second period of time. For example, after the battery 101 is turned on, a power output of the battery may initially increase from zero to a first power and then may remain constant at the first power.

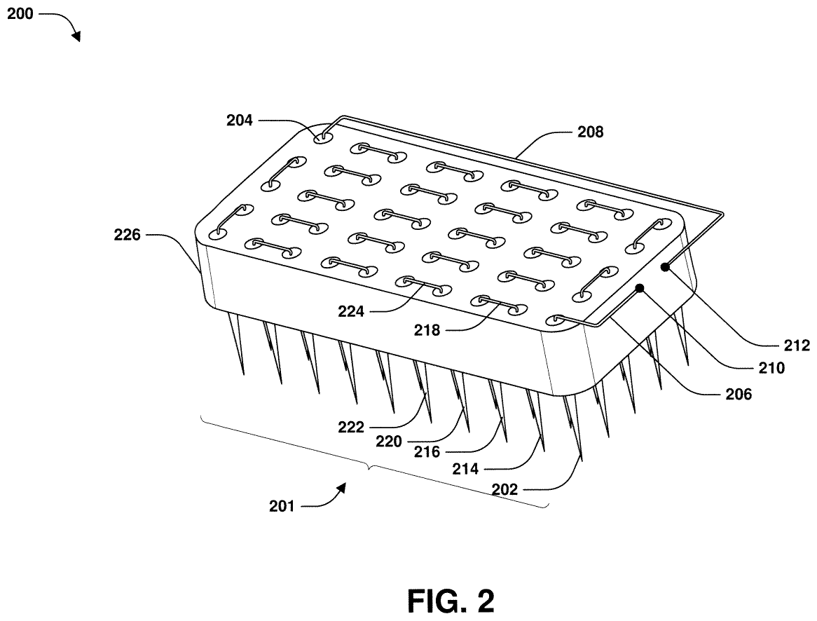

FIG. 2 illustrates a needle patch battery 200, according to some embodiments. In some examples, the needle patch battery 200 may comprise a body 226, a plurality of needles 201 (e.g., a plurality of electrodes) and a set of electrical conductors. In some examples, the body 610 may comprise one or more materials that are electrically insulating. In some examples, the plurality of needles 201 may be a plurality of electrodes. In some examples, the plurality of electrodes may be a plurality of anodes and/or a plurality of cathodes. For example, a needle 202, a needle 216 and/or a needle 222 are anodes while a needle 214 and/or a needle 220 are cathodes. In some examples, an anode of the plurality of needles 201 may be surrounded by at least one cathode.

In some examples, a cathode of the plurality of needles 201 may be surrounded by at least one anode. In some examples, the set of conductors may comprise a first set of conductors and/or a second set of conductors. In some examples, a first conductor of the second set of conductors may be connected to an anode (e.g., the needle 202) of the plurality of needles 201 and may be a positive terminal of the needle patch battery 200. In some examples, the first conductor of the second set of conductors may be connected to the anode (e.g., the needle 202) of the plurality of needles 201 and may be a negative terminal of the needle patch battery 200.

In some examples, a second conductor of the second set of conductors may be connected to the cathode (e.g., a needle 204) and may be a negative terminal of the needle patch battery 200. In some examples, the second conductor of the second set of conductors may be connected to a cathode (e.g., the needle 204) and may be a positive terminal of the needle patch battery 200.

In some examples, each conductor of the first set of conductors may connect an anode of the plurality of anodes to a cathode of the plurality of cathodes. For examples, a conductor 218 of the second set of conductors may connect the needle 214 (e.g., a cathode) to the needle 216 (e.g., an anode) and/or a conductor 224 of the second set of conductors may connect the needle 220 (e.g., a cathode) to the needle 222 (e.g., an anode). In some examples, a first needle of the plurality of needles 201 may be a first electrode and may be made of a first material. In some examples, a second needle of the plurality of needles 201 may be a second electrode and may be made of a second material. In some examples, instead of using usual polymer needles in injection patches, the plurality of needles 201 are made of specific metals.

In some examples, the needle patch battery 200 may comprise a conductor 206, wherein the conductor 206 may be connected to the needle 202 on one side and the other side may be a positive terminal or a negative terminal of the needle patch battery 200. In some examples, the needle patch battery 200 may comprise a conductor 208, wherein the conductor 208 may be connected to the needle 204 on one side and the other side may be a positive terminal or a negative terminal of the needle patch battery 200. In some examples, the conductor 206 may comprise a head 210 and/or the conductor 208 may comprise a head 212. In some examples, the head 210 may be a positive terminal of the needle patch battery 200 and/or the head 212 may be a negative terminal of the needle patch battery 200. In some examples, the head 210 may be a negative terminal of the needle patch battery 200 and/or the head 212 may be a positive terminal of the needle patch battery 200.

In some examples, the plurality of needles 201 may penetrate a body (e.g., a human body, a body of an animal, etc.) and use one or more electrolytes inside the body. In some examples, the one or more electrolytes may comprise one or more bodily fluids. The one or more bodily fluids may comprise a blood (e.g., a human blood, an animal blood, etc.), a cerebrospinal fluid (e.g., a human cerebrospinal fluid, an animal cerebrospinal fluid, etc.), a urine (e.g., a human urine, an animal urine, etc.), a body sweat (e.g., a human body sweat, an animal body sweat, etc.), a peritoneal fluid (e.g., a human peritoneal fluid, an animal peritoneal fluid, etc.) and/or an interstitial fluid (e.g., a human interstitial fluid, an animal interstitial fluid, etc.). In some examples, the plurality of needles 201 may be placed on a skin of the body and use body sweat as an electrolyte. In some examples, an anode (e.g., the needle 202) of the plurality of needles 201 and/or a cathode (e.g., the needle 204) of the plurality of needles 201 may undergo electrochemical reactions with the one or more electrolytes to generate electrical energy for the one or more electrical devices.

In some examples, the plurality of needles 201 may be made of Copper, Brass, Platinum, Gold, Silver, Palladium, Lithium, Graphite, Zinc, Aluminum, Iron and/or Nickel.

In some examples, at least a portion of the plurality of needles 201 in the needle patch battery 200 may be covered with a magnesium material to prevent corrosion of the plurality of electrodes and to maintain conductivity and electron transfer of the plurality of electrodes.

In some examples, the plurality of needles 201 may enter a container (e.g., a housing) and use one or more second electrolytes inside the container. In some examples, the one or more second electrolytes may comprise one or more natural materials. The one or more natural materials may comprise glucose, standard white vinegar, liquid copper ion, liquid iron ion, liquid zinc ion, fenugreek plant extract, melted white chocolate, liquid oil, flower petal water with bacteria, ice cream, heated and melted ice cream, dried soil, soil with water (hard clay), soil with water (very thin clay), cooked potato puree, lemon puree, sheep blood, peritoneal fluid and/or cerebrospinal fluid. In some examples, the one or more natural materials may comprise one or more natural enzymes.

In some examples, the needle patch battery 200 may generate an ascending power in a first period of time and a constant power in a second period of time. For example, after the needle patch battery 200 is turned on, power output of the needle patch battery 200 may initially increase from zero to a first power and then may remain constant at the first power.

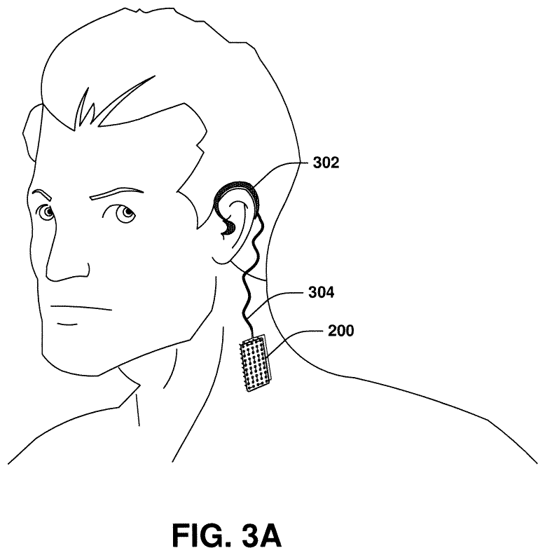

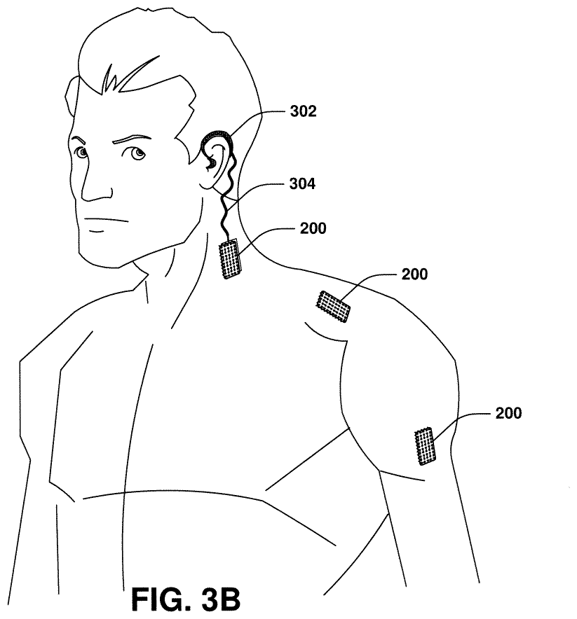

FIG. 3A illustrates the needle patch battery 200 attached to a person's neck, and a connecting wire 304 connects it to a hearing aid 302, according to some embodiments. In some examples, the needle patch battery 200 may be used for transdermal delivery (e.g., skin injection) and/or the connecting wire 304 may connect the needle patch battery 200 of the present disclosure to a battery compartment in the hearing aid 302. In some examples, the hearing aid 302 may be placed inside an ear of a deaf person (e.g., a patient).

FIG. 3B illustrates a plurality of needle patch batteries of the present disclosure, wherein the plurality of needle patch batteries are attached to various areas of a body of a patient.

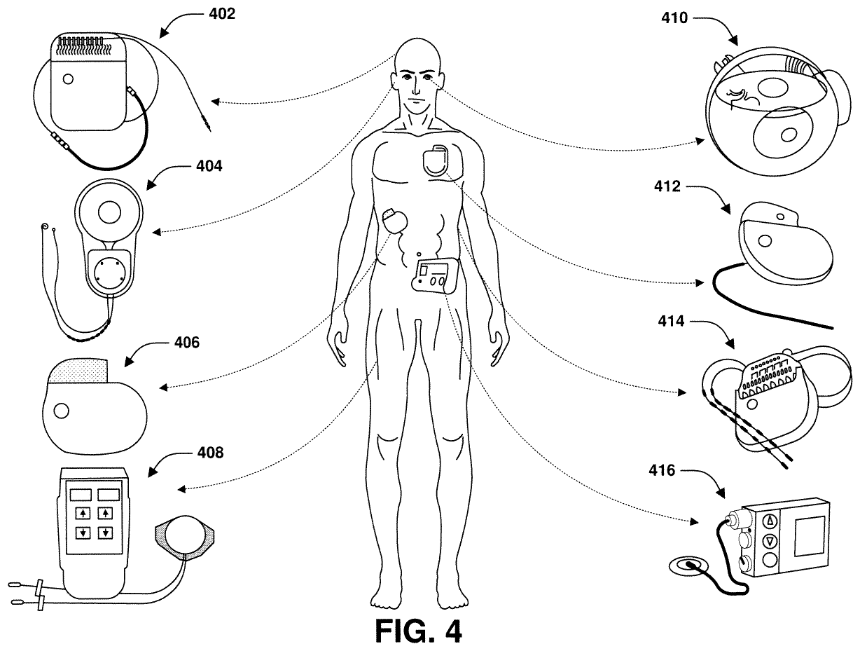

FIG. 4 illustrates a plurality of medical applications of the biological battery (e.g., the battery 101, the needle patch battery 200) of the present disclosure within a plurality of implantable medical devices. In some examples, the plurality of implantable medical devices are a deep brain neuro stimulator 401, a gastric stimulator 402, a foot drop stimulator 403, a cochlear implant 404, a retinal implant 405, a cardiac defibrillator pacemaker 406, a spinal cord stimulator 407 and/or an insulin pump 408.

In some examples, the biological battery of the present disclosure may increase contact surface of anodic electrodes and/or cathodic electrodes without using a composite or deposit special compounds on the surface of the electrodes.

In some examples, the biological battery of the present disclosure may be an inexpensive battery because of not using gold nanowires, gold nanoparticles, platinum nanowires, platinum nanoparticles and/or carbon nanotubes.

In some examples, the biological battery of the present disclosure may be a simple-design battery because of not using special catalysts for oxidation-reduction reactions, specific enzymes, enzyme immobilization, dipeptides and/or diphenylalanine-silver composites.

In some examples, designing and arrangement of the electrodes of the biological battery of the present disclosure is novel. In some examples, the biological battery of the present disclosure may not need to be replaced and/or charged.

In some examples, the biological battery is utilized in one or more types of technical filed. The one or more types of technical filed may be medical engineering, medical equipment, manufacturing of smart bio-hearing aids, electronics, chemical and electrical energy storage, enzyme-based bio-fuel cells, nanotechnology and/or nano-biotechnology.

The biological battery of the present disclosure may be utilized in all electrical devices requiring batteries. In some examples, the biological battery of the present disclosure may be utilized in hearing aids and may attempt to highlight the significant points of replacing this kind of battery in hearing aids and its advantages. For example, after providing a hearing aid, a battery is required for its use. In some examples, bases upon depletion of a battery, a hearing aid may not work well. Replacing hearing aid batteries is one of the major problems and discomforts for users of this device. Approximately fifty million people worldwide use hearing aids, which equates to an annual consumption of two billion zinc-air batteries by these users. Due to the need to charge batteries, especially heart batteries, which may result in patient mortality during surgery, there is a critical need for a long-lasting battery that can endure for an extended period. One of technical Problems in current hearing aids is premature battery depletion. Current hearing aid batteries typically last only 2-3 days, requiring frequent replacements. Most of current hearing aid batteries are non-rechargeable, leading to disposal and the need for frequent purchases. Incompatible batteries can introduce noise and distort sound quality. Elderly, children, and infants may struggle to change current batteries independently. Based upon lack of awareness, young children and infants may not notice when their hearing aid battery is depleted. Changing batteries is a time-consuming process. Incompatibility between batteries and hearing aids can introduce noise and distortion. Environmental factors such as humidity and temperature fluctuations may adversely affect performance of current batteries.

In some examples, the biological battery of the present disclosure may be an implantable biological battery. In some examples, the biological battery of the present disclosure may enhance convenience for users. In some examples, the biological battery of the present disclosure may improve sound quality. In some examples, the biological battery of the present disclosure may comprise a high energy efficiency. In some examples, the biological battery of the present disclosure may be a long-life battery. In some examples, the biological battery of the present disclosure may simplify usage and/or may be a versatile battery. In some examples, the biological battery of the present disclosure may be used in a hearing aid, wherein the biological battery may be implanted into body of a patient and may use one or more bodily fluids inside the body of the patient. In some examples, the biological battery of the present disclosure in one or more electrical devices (e.g., a hearing aid) may provide a solution for individuals, especially the elderly, children, and infants, who may have difficulty changing batteries. In an example, the biological battery of the present disclosure in a hearing aid may eliminate noise and distortion caused by other incompatible batteries. In some examples, the biological battery of the present disclosure is utilized to provide sufficient power for extended periods. In some examples, the biological battery may last for years, potentially a lifetime, without requiring replacement. In some examples, usage of the biological battery of the present disclosure is simple and the biological battery of the present disclosure may reduce a risk of complications for its users. In some examples, the biological battery of the present disclosure may be used in a wide range of electrical devices, including mobile chargers, tablets, laptops, and/or medical devices, wherein the medical devices may be cochlear implants, pacemakers, artificial hearts, insulin pumps, drug pumps, retinal implants, and/or nerve stimulators.

In some examples, the biological battery of the present disclosure may eliminate surgery and implanting a battery within blood vessels. In some examples, the biological battery of the present disclosure may be easily attached to a patient's skin and may use the person's bodily fluids (e.g., blood). In some examples, unlike previous implants and surgical procedures to place a component or microchip within a patient's body, the biological battery of the present disclosure may be placed on a desired location of the patient's skin or body and may be easily replaced.

In some examples, the creation and construction of the biological battery of the present disclosure are straightforward and may not require any complex procedures. The biological battery of the present disclosure may leverage electrochemical reactions that occur naturally and may release energy. In some examples, the biological battery of the present disclosure may utilize the same natural substances found within the body, such as blood, while other references employ batteries with toxic chemicals enclosed in casings to prevent contact with the body, wherein such contact can corrode the battery, damage the casing, and/or release harmful substances into the body. In some examples, the biological battery of the present disclosure may use the individual's bodily fluids (e.g., blood), ensuring full biocompatibility and eliminating the need for chemical electrolytes.

In some examples, using the biological battery of the present disclosure may eliminate the need for a skilled surgeon, the risk of vascular rupture during surgery, and the long-term presence of a foreign object within the body, which may lead to complications such as vascular occlusion. In some examples, at least a portion of the biological battery (e.g., a plurality of needle patches) of the present disclosure may be inserted into a blood vessel may eliminate the need for surgery and may reduce the risk of complications such as infection and vascular rupture. In some examples, the needle patches (e.g., the needle patches that are currently used as an alternative to injections) may cause minimal pain or skin damage. In some examples, using the needle patches in the biological battery of the present disclosure may be less invasive and less risky than applying complex surgeries to implant a battery within blood vessels. In some examples, the biological battery of the present disclosure may be user-friendly, may reduce many risks for a patient and/or may also lower costs.

In some examples, the plurality of needle patches may be a plurality of microneedle patches, wherein using the plurality of microneedle patches may be used in adhesive bandages and/or decorative stickers. In some examples, number of needles (e.g., microneedles) in the plurality of needle patches may vary depending on battery's requirements and the desired voltage output of the biological battery.

In some examples, the biological battery of the present disclosure may generate higher voltage output compared to other tested references. In some examples, the biological battery of the present disclosure may allow for easy generation of a high voltage (e.g., a voltage range between 0.1 to 25 volts) without the need for any amplifiers or energy storage devices. In some examples, the biological battery of the present disclosure may allow for easy generation of a high voltage (e.g., a voltage range between 2 to 12 volts) without the need for any amplifiers or energy storage devices.

In some examples, the biological battery of the present disclosure may not use Nafion membranes, coatings, carbon nanotubes, various nanoparticles, and specific enzymes on electrodes.

In some examples, the biological battery of the present disclosure may be a battery that may not rely on numerous nanoparticles, materials, membranes, enzymes, or coating methods.

In some examples, the voltage and amperage produced by the biological battery of the present disclosure may significantly higher than those reported in previous references, making it easy to power electronic devices.

In some examples, manufacturing process of the biological battery of the present disclosure may involve cutting selected metals into appropriate pieces and then connecting them in pairs using thin wires. These pairs are then placed in dedicated compartments. Each compartment may be filled with a desired electrolyte.

In some examples, a very small amount of blood, two types of metals for cutting and creating electrodes, containers (e.g., a housing assembly) that can be made of metal, glass, or plastic, and/or thin wires (e.g., wires of telephone cables) may be provided to produce the biological battery of the present disclosure.

In some examples, the biological battery may be a membraneless battery because of not using a middle membrane (e.g., a Nafion membrane or a composite membrane similar to Nafion membrane).

In some examples, using the biological battery of the present disclosure may reduce health risks. In some examples, the biological battery of the present disclosure may eliminate the risk of post-surgical infections. In some examples, the biological battery of the present disclosure may eliminate the risk of infections caused by a foreign object incompatible with the body's nature within a blood vessel. In some examples, the biological battery of the present disclosure may eliminate the risks associated with bleeding due to vascular rupture during or after surgery to implant a device within a blood vessel. In some examples, the biological battery of the present disclosure may eliminate the need for strong antibiotics to prevent infections caused by a foreign object within a blood vessel and after surgery. In some examples, using the biological battery of the present disclosure may reduce treatment time and duration.

In some examples, the biological battery of the present disclosure may eliminate the costs of post-operative medications. In some examples, since the biological battery of the present disclosure operates based upon naturally occurring reactions, it may require minimal manufacturing costs.

In some examples, the biological battery of the present disclosure may (i) not use carbon nanotubes, which are highly toxic and expensive, (ii) eliminate the use of expensive membranes like Nafion, (iii) be easily attached to the skin and uses the person's blood, eliminating the need for surgery, (iv) provide a significantly higher voltage compared to other references, allowing for powering various medical devices without the need for additional power sources, and (v) be easily manufactured using natural and inexpensive materials.

In some examples, the biological battery of the present disclosure may (i) solve all the problems associated with traditional batteries, and (ii) require no charging or replacement.

In some examples, the biological battery of the present disclosure may utilize an adhesive patch containing microscopic needles that, upon contact with the inner layer of a skin, establish a connection with the body's blood. In some examples, the microscopic needles may be biocompatible and may not harm the body. In some examples, the microscopic needles may be selected depending on choosing a type of electrodes. For example, if an aluminum electrode is required, an aluminum needle may be used, and if a copper electrode is required, a copper needle may be used. In some examples, the microscopic needles may be coated by anti-corrosion materials (e.g., magnesium metal) to prevent corrosion of the electrodes (e.g., the microscopic needles) and enhance conductivity of the electrodes.

In some examples, the biological battery of the present disclosure may have an ability to adjust the voltage and power output for each electrical device by changing the electrolytes and electrodes.

In some examples, the biological battery of the present disclosure may be an enzymatic biological battery. In some examples, upon an innovative design, the biological battery of the present disclosure may produce a wide range of voltages, allowing users to adjust the output to match the specific requirements of their electrical devices.

In some examples, unlike traditional methods, the biological battery of the present disclosure may not rely on nanomaterials like gold nanoparticles, carbon nanotubes, and/or graphene. In some examples, the biological battery of the present disclosure may not use the nanomaterials like the gold nanoparticles, the carbon nanotubes, and/or the graphene.

In some examples, the biological battery of the present disclosure may use natural enzymes found in blood or other electrolytes. In some examples, the biological battery may eliminate the need for artificial enzymes or enzyme immobilization.

In some examples, a battery is provided. The battery includes a plurality of electrodes, a first set of electrical conductors configured to connect one or more of the plurality of electrodes pairwise, a second set of electrical conductors configured to connect the plurality of electrodes to an electrical device, and a housing assembly configured to house an electrolyte and a set of electrodes of the plurality of electrodes.

In some examples, the housing assembly includes a plurality of housings, wherein a first housing of the plurality of housings is configured to house a first electrolyte, a first anode of the plurality of electrodes and a first cathode of the plurality of electrodes, and a second housing of the plurality of housings is configured to house a second electrolyte, a second anode of the plurality of electrodes and a second cathode of the plurality of electrodes.

In some examples, an electrical conductor of the first set of electrical conductors is configured to connect the first cathode to the second anode. In some examples, a first electrical conductor of the second set of electrical conductors is a positive terminal of the battery. In some examples, a second electrical conductor of the second set of electrical conductors is a negative terminal of the battery.

In some examples, an anode of the plurality of electrodes and a cathode of the plurality of electrodes undergo electrochemical reactions with the electrolyte in the housing assembly to generate electrical energy for the electrical device.

In some examples, the battery includes an inlet port configured to receive the electrolyte and conduct the electrolyte to the housing assembly.

In some examples, the electrical device includes a mobile charger, a mobile phone, a tablet, a laptop, and/or a medical device wherein the medical device includes a hearing aid, a cochlear implant, a pacemaker, an artificial heart, an insulin pump, a drug pump, a retinal implant, and/or a nerve stimulator.

In some examples, the plurality of electrodes include an anode including Copper, Brass, Platinum, Gold, Silver, Palladium, Lithium, Graphite, Zinc, Aluminum, Iron, and/or Nickel, and a cathode including Copper, Brass, Platinum, Gold, Silver, Palladium, Lithium, Graphite, Zinc, Aluminum, Iron, and/or Nickel.

In some examples, a magnesium metal covers at least a portion of the plurality of electrodes to prevent corrosion of the plurality of electrodes and to maintain conductivity and electron transfer of the plurality of electrodes.

In some examples, a voltage generated by the battery is between about 0.1 volt to about 25 volts, and/or between about 2 volts to about 12 volts.

In some examples, the electrolyte includes a bodily fluid, wherein the bodily fluid includes a blood, a cerebrospinal fluid, a urine, a body sweat, a peritoneal fluid, and/or an interstitial fluid.

In some examples, the electrolyte includes a natural material containing natural enzymes, wherein the natural material includes glucose, standard white vinegar, liquid copper ion, liquid iron ion, liquid zinc ion, fenugreek plant extract, melted white chocolate, liquid oil, flower petal water with bacteria, ice cream, heated and melted ice cream, dried soil, soil with water (hard clay), soil with water (very thin clay), cooked potato puree, lemon puree, sheep blood, peritoneal fluid, and/or cerebrospinal fluid.

In some examples, the battery is an enzymatic fuel cell (e.g., an enzymatic bio-fuel cell).

In some examples, a battery is provided. The battery includes a patch including a plurality of needles configured to interface with a body, a first set of electrical conductors configured to connect one or more of the plurality of needles pairwise, and a second set of electrical conductors configured to connect the plurality of needles to an electrical device.

In some examples, the patch is configured to attach to an outer layer of a body skin.

In some examples, the plurality of needles are configured to penetrate a body to be in contact with a bodily fluid.

In some examples, the bodily fluid includes a blood, a cerebrospinal fluid, a urine, a body sweat, a peritoneal fluid, and/or an interstitial fluid.

In some examples, a magnesium metal covers at least a portion of the plurality of needles to prevent corrosion of the plurality of needles.

In some examples, the plurality of needles includes an anode including Copper, Brass, Platinum, Gold, Silver, Palladium, Lithium, Graphite, Zinc, Aluminum, Iron, and/or Nickel, and a cathode including Copper, Brass, Platinum, Gold, Silver, Palladium, Lithium, Graphite, Zinc, Aluminum, Iron, and/or Nickel.

In some examples, a battery is provided. The battery includes a plurality of electrodes configured to be in contact with one or more electrolytes, a first set of electrical conductors configured to connect one or more of the plurality of electrodes pairwise, and a second set of electrical conductors configured to connect the plurality of electrodes to an electrical device.

In some examples, the electrolyte includes a bodily fluid, and/or a natural material containing natural enzymes.

Unless specified otherwise, “first,” “second,” and/or the like are not intended to imply a temporal aspect, a spatial aspect, an ordering, etc.

Rather, such terms are merely used as identifiers, names, etc. for features, elements, items, etc. For example, a first object and a second object generally correspond to object A and object B or two different or two identical objects or the same object.

Moreover, “example” is used herein to mean serving as an instance, illustration, etc., and not necessarily as advantageous. As used herein, “or” is intended to mean an inclusive “or” rather than an exclusive “or”. In addition, “a” and “an” as used in this application are generally be construed to mean “one or more” unless specified otherwise or clear from context to be directed to a singular form. Also, at least one of A and B and/or the like generally means A or B or both A and B. Furthermore, to the extent that “includes”, “having”, “has”, “with”, and/or variants thereof are used in either the detailed description or the claims, such terms are intended to be inclusive in a manner similar to the term “comprising”.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing at least some of the claims.

Various operations of embodiments and/or examples are provided herein. The order in which some or all of the operations are described herein should not be construed as to imply that these operations are necessarily order dependent. Alternative ordering will be appreciated by one skilled in the art having the benefit of this description. Further, it will be understood that not all operations are necessarily present in each embodiment and/or example provided herein. Also, it will be understood that not all operations are necessary in some embodiments and/or examples.

Also, although the disclosure has been shown and described with respect to one or more implementations, equivalent alterations and modifications will occur to others skilled in the art based upon a reading and understanding of this specification and the annexed drawings. The disclosure includes all such modifications and alterations and is limited only by the scope of the following claims. In particular regard to the various functions performed by the above described components (e.g., elements, resources, etc.), the terms used to describe such components are intended to correspond, unless otherwise indicated, to any component which performs the specified function of the described component (e.g., that is functionally equivalent), even though not structurally equivalent to the disclosed structure. In addition, while a particular feature of the disclosure may have been disclosed with respect to only one of several implementations, such feature may be combined with one or more other features of the other implementations as may be desired and advantageous for any given or particular application.

What is claimed:

1. A battery comprising:
a plurality of electrodes;
a first set of electrical conductors configured to connect one or more of the plurality of electrodes pairwise;
a second set of electrical conductors configured to connect the plurality of electrodes to an electrical device; and
a housing assembly configured to house an electrolyte and a set of electrodes of the plurality of electrodes.

2. The battery of claim 1, wherein:
the housing assembly comprises a plurality of housings, wherein:
a first housing of the plurality of housings is configured to house a first electrolyte, a first anode of the plurality of electrodes and a first cathode of the plurality of electrodes; and
a second housing of the plurality of housings is configured to house a second electrolyte, a second anode of the plurality of electrodes and a second cathode of the plurality of electrodes.

3. The battery of claim 2, wherein:
an electrical conductor of the first set of electrical conductors is configured to connect the first cathode to the second anode;
a first electrical conductor of the second set of electrical conductors is a positive terminal of the battery; and
a second electrical conductor of the second set of electrical conductors is a negative terminal of the battery.

4. The battery of claim 1, wherein:
an anode of the plurality of electrodes and a cathode of the plurality of electrodes undergo electrochemical reactions with the electrolyte in the housing assembly to generate electrical energy for the electrical device.

5. The battery of claim 1, comprising:
an inlet port configured to receive the electrolyte and conduct the electrolyte to the housing assembly.

6. The battery of claim 1, wherein the electrical device comprises at least one of:
a mobile charger;
a mobile phone;
a tablet;
a laptop; or
a medical device wherein the medical device comprises at least one of:
a hearing aid;
a cochlear implant;
a pacemaker;
an artificial heart;
an insulin pump;
a drug pump;
a retinal implant; or
a nerve stimulator.

7. The battery of claim 1, wherein:
the plurality of electrodes comprise:
an anode comprising at least one of:
Copper;
Brass;
Platinum;
Gold;
Silver;
Palladium;
Lithium;
Graphite;
Zinc;
Aluminum;
Iron; or
Nickel; and
a cathode comprising at least one of:
Copper;
Brass;
Platinum;
Gold;
Silver;
Palladium;
Lithium;
Graphite;
Zinc;
Aluminum;
Iron; or
Nickel.

8. The battery of claim 7, wherein a magnesium metal covers at least a portion of the plurality of electrodes to prevent corrosion of the plurality of electrodes and to maintain conductivity and electron transfer of the plurality of electrodes.

9. The battery of claim 1, wherein a voltage generated by the battery is at least one of:
between about 0.1 volt to about 25 volts; or
between about 2 volts to about 12 volts.

10. The battery of claim 1, wherein:
the electrolyte comprises a bodily fluid, wherein the bodily fluid comprises at least one of:
a blood;
a cerebrospinal fluid;
a urine;
a body sweat;
a peritoneal fluid; or
an interstitial fluid.

11. The battery of claim 1, wherein:
the electrolyte comprises a natural material containing natural enzymes, wherein the natural material comprises at least one of:
glucose;
standard white vinegar;
liquid copper ion;
liquid iron ion;
liquid zinc ion;
fenugreek plant extract;
melted white chocolate;
liquid oil;
flower petal water with bacteria;
ice cream;
heated and melted ice cream;
dried soil;
soil with water (hard clay);
soil with water (very thin clay);
cooked potato puree;
lemon puree;
sheep blood;
peritoneal fluid; or
cerebrospinal fluid.

12. The battery of claim 1, wherein the battery is an enzymatic fuel cell.

13. A battery comprising:
a patch comprising a plurality of needles configured to interface with a body;
a first set of electrical conductors configured to connect one or more of the plurality of needles pairwise; and
a second set of electrical conductors configured to connect the plurality of needles to an electrical device.

14. The battery of claim 13, wherein:
the patch is configured to attach to an outer layer of a body skin.

15. The battery of claim 13, wherein:
the plurality of needles are configured to penetrate the body to be in contact with a bodily fluid. The battery of claim 15, wherein the bodily fluid comprises at least one of:
a blood;
a cerebrospinal fluid;
a urine;
a body sweat;
a peritoneal fluid; or
an interstitial fluid.

17. The battery of claim 13, wherein:
a magnesium metal covers at least a portion of the plurality of needles to prevent corrosion of the plurality of needles.

18. The battery of claim 13, wherein:
the plurality of needles comprise:
an anode comprising at least one of:
Copper;
Brass;
Platinum;
Gold;
Silver;
Palladium;
Lithium;
Graphite;
Zinc;
Aluminum;
Iron; or
Nickel; and
a cathode comprising at least one of:
Copper;
Brass;
Platinum;
Gold;
Silver;
Palladium;
Lithium;
Graphite;
Zinc;
Aluminum;
Iron; or
Nickel.

19. A battery comprising:
a plurality of electrodes configured to be in contact with one or more electrolytes;
a first set of electrical conductors configured to connect one or more of the plurality of electrodes pairwise; and
a second set of electrical conductors configured to connect the plurality of electrodes to an electrical device.

20. The battery of claim 19, wherein:
the electrolyte comprises at least one of:
a bodily fluid; or
a natural material containing natural enzymes.
